(19) United States
(12) Patent Application Publication
(10) Pub. No.: US 2011/0135627 A1
(43) Pub. Date: Jun. 9, 2011

(54) PAIN RELIEF COMPOSITION, SYSTEM AND METHOD

(75) Inventors: Richard LaMotta, Mount Kisco, NY (US); Robert Ornstein, Mohegan Lake, NY (US); Douglas Redfield, Mount Kisco, NY (US); Vijay Vad, New York, NY (US)

(73) Assignee: The Inflasoothe Group, Inc., Mount Kisco, NY (US)

(21) Appl. No.: 12/900,069

(22) Filed: Oct. 7, 2010

Related U.S. Application Data
(60) Provisional application No. 61/249,450, filed on Oct. 7, 2009.

(51) Int. Cl. A61K 38/48; A61K 31/165; A61P 29/00

(52) U.S. Cl. 424/94.65; 514/627

(57) ABSTRACT

A composition for the relief of pain includes comprising a transducer, a fibrinolytic, at least one of a pain relief agent or an anti-inflammatory agent. The pain relief agent can be capsaicin and the fibrinolytic can be bromelain. The composition can also include at least one of a cooling component, a circulation increasing component, a joint or muscle soothing component and a muscle membrane stabilizer. the cooling agent fcan be menthol. A cream composition can include at least one cetyl esterified fatty acid. A cream can be made by combining the oil with a cream base. Suitable compositions include creams, gels, salves, sprays, powder, balms, liniment, ointments, and transdermal patches A system for pain relief that includes the oil and the cream, or a kit containing a container of the oil and a container of the cream.

### CROSS-REFERENCE TO RELATED APPLICATIONS

This application claims priority to U.S. provisional application No. 61/249450 filed Oct. 7, 2009, which is incorporated herein by reference in its entirety.

### BACKGROUND OF THE INVENTION

1. Field of the Invention

The present invention relates generally to a formulation for ameliorating pain. More particularly, the invention is a composition that includes a transducer, a fibrinolytic and at least one pain relief agent or anti-inflammatory agent. The composition can include a combination of natural clinically proven natural extracts with anti-inflammatory properties, substance P blockers, muscle membrane stabilizers, and cetyl esterified fatty acids. The present invention relates generally to a joint and pain formula and more particularly to a unique and optimized blending with ingredients combined into one single formula to promote healing and relieve pain symptoms.

2. Background

Pain is a disease of epidemic proportions and is thought to affect more than 150 million Americans, costing the American public alone approximately $100 billion each year in health care, compensation, and litigation (Loeser, J. D. Butler, S. H. Chpaman, C. R. & Turk, K. C., eds, (2001) Bonica's Management of Pain (Lippincott, Philadelphia)). It is generally accepted to be the leading cause of joint movement limitations and disability while receiving a great deal of research and medical attention. Over one-third of the world's population suffers from persistent or recurrent pain.

Chronic pain is associated with conditions such as back injury, migraine headaches, arthritis, herpes zoster, diabetic neuropathy, temporomandibular joint syndrome, and cancer. Many of the currently available pain therapies are either inadequate or cause uncomfortable to deleterious die effects. Chronic pain results not just from the physical insult but also from a combination of physical, emotional, psychological, and social abnormalities. Because pain often persists in many individuals even after an insult is healed, it is the ongoing pain rather than the injury underlies the patient's disability. Untreated pain may become self-perpetuating because pain has immunosuppressive effects that leave patients susceptible to subsequent diseases.

Current therapies all have limitations. Opinoids can cause tolerance, dependence, constipation, respiratory depression and sedation. NSAIDS have gastrointestinal side effects, can increase bleeding time, and are not effective in the treatment of severe pain. In the case of non-selective sodium channel blockers, central nervous system (CNS) side effects, cardiovascular side effects and corneal damage have been reported after use. Given the above limitations to currently known pain-control therapies, a need still exists for better pain-treatment methods. Many products available include expensive ingredients, require frequent applications and provide relief of pain for only short periods. Many products that provide pain relief do not enhance recovery.

There is a need for further pain relieving formulations or formulations that enhance the recovery from pain. Natural extracts may impart not only pleasant odor but also medicinal properties useful in healing wounds or muscular injuries, such as anti-inflammatory properties, antibiotic properties, deodorizing properties or muscle relaxing properties. There are many topical treatments for pain some treatments are available over the counter at pharmacies and or supermarkets others are prescription only medicines.

### SUMMARY OF THE INVENTION

In some embodiments, the present invention is a composition that can include a transducer, a fibrinolytic and at least one of a pain relief agent or an anti-inflammatory agent. The composition can further include at least one of a cooling component, a circulation increasing component, a joint or muscle soothing component and a muscle membrane stabilizer. Exemplary compositions can include capsaicin as a pain relief agent, bromelain as a fibrinolytic and/or menthol as a cooling agent. The composition can be in the form of an oil, a cream, a gel, a salve, a spray, powder, balm, liniment, or ointment. It can also be applied to a delivery system such as a wipe, adhesive strip or transdermal patch. In some embodiments, the composition is a cream that includes a blend of cetyl esterified fatty acids.

In other embodiments, the invention is a pain relief system that includes more than one component, for example an oil and a cream. The oil can include a transducer, a fibrinolytic, at least one of a pain relief agent or an anti-inflammatory agent and, optionally, at least one of a cooling component, a circulation increasing component, a joint or muscle soothing component, and a muscle membrane stabilizer. The cream can include cetyl esterified fatty acids (also referred to herein as cetylated fatty acids), and emulsifiers, surfactants and/or solubilizing agents. In exemplary embodiments, in addition to cetylated fatty acids, the cream includes a pain relief agent and/or a cooling agent.

There are a number of methods for using the system for pain relief and other purposes. In general, however, the components are applied topically. For example, the cream alone may be applied or the oil alone may be applied. Alternatively, the cream can be applied initially with one or more applications of the oil over the cream. In exemplary embodiments, the method of use includes topical application of the cream to the area experiencing pain. If pain persists, the oil is applied on top of the cream, and the application of oil repeated if pain continues. If the combination of cream and multiple applications of oil is insufficient to relieve pain, the oil can be used alone.

The present invention is thus a topical pain relief formulation that can also have anti-inflammatory effects. Exemplary embodiments include a combination of natural ingredients that absorb into the skin rapidly providing fast, safe, effective, long lasting pain relief. The invention is unique and distinguishable from existing formulations and includes a process for incorporating natural extracts combined with cetylated fatty acids that has not been previously achieved or used.

The presence of fibrinolytic ingredients and transducers in a unique combination can provide for penetration of the ingredients through multiple layers of skin, fat and muscle to provide healing, ant-inflammatory, and/or pain relieving effects. Exemplary embodiments include one or more pain relief components and one or more anti-inflammatory agents. Other ingredients can be added to provide additional beneficial properties.

Exemplary embodiments of the invention can include one or more of anti-inflammatory agents such as oils, fibrinolytics, pain relief agents, such as Substance P blockers, muscle membrane stabilizers, and cetyl esterified fatty acids. Exemplary anti-inflammatory oils include, for example, wintergreen, ginger oil, blends of emu oil, menthol, arnica, cetyl esterified fatty acids. Exemplary fibrinolytics, include, for example, bromelain. Exemplary transducers include, for example, a blend of emu oil and aloe vera, and can include cetylated fatty acids. Solvents can also function as tranducers in some circumstances. Exemplary pain relief agents include Substance P blockers such as, for example capsaicin. Exemplary muscle membrane stabilizers include magnesium compounds. Exemplary cetyl esterified fatty acids include, for example, cetyl laureate, cetyl lineoleate, cetyl myristate, cetyl myristoleate, cetyl oleate, cetyl palmitate, and cetyl palmitoleate. Cetyl esterified fatty acids are clinically proven anti-inflammatory cell membrane lubricants. An exemplary solvent for the oil composition is dimethyl sulfoxide.

Other exemplary embodiments include one or more of a pain relief component, an anti-inflammatory component, a cooling component, a circulation increasing component, a joint or muscle soothing component. In some embodiments, the invention is a method of making a pain relief composition comprising preparing an oil, separately preparing a cream base and combining the oil with the cream base to form a cream. The oil can include a transducer, a fibrinolytic, at least one of a pain relief agent or an anti-inflammatory agent and a solvent. The cream base can include at least one cetyl esterified fatty acid and an emulsifier or surfactant. The cream base can also include a cooling agent. In exemplary embodiments, the oil and the cream base each comprise a pain relief agent and/or a cooling agent and the concentration of pain relief agent and/or the cooling agent is about the same in both the cream base and the same.

In still other embodiments, the invention is a pain relief system that includes an oil and a cream, wherein the oil includes a transducer, a fibrinolytic, at least one of a pain relief agent or an anti-inflammatory agent and a solvent and the cream includes at least one cetyl esterified fatty acid and an emulsifier or surfactant. In such embodiments, the invention can be in the form of a kit that includes a first container containing the oil and a second container containing the cream.

While some components used in compositions according to the present invention have been known to demonstrate health benefits when administered individually, the present invention relates to novel combinations of natural compounds that demonstrate the properties of the compositions when administered as specified combinations. In general, the specific compositions of the present invention exhibit synergistic enhancement of their efficacies when administered in combination. Suitable natural extracts for use in the invention include Aloe Vera, Boswella extract, arnica extract, methyl salicylate oil, wintergreen, devils claw, cats claw, burdock root, tea tree, lavender, willow bark and others.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

Unless specified otherwise, the use of the term percent, percentage or % refers to a percent by weight (% w/w). The terms “component” or “agent” are generally used interchangeably herein unless context dictates otherwise. Topical application includes rubbing, spraying or other methods that provide placement on the surface of the skin.

In some embodiments, the invention is a topical anti-inflammatory pain reliever that contains a combination of natural ingredients that absorb into the skin rapidly providing fast, safe, effective, long lasting pain relief. The composition is designed to slow and/or reverse the effects of pain and inflammation in any muscle group, thus improving mobility and overall quality of life. In exemplary embodiments, blends of the high quality, standardized ingredients work together in a cream to provide a topical treatment that penetrates deeply into the skin, delivering components, such as herbal ingredients and other nutrients and actives, directly into the painful, inflamed areas. The topical composition penetrates through the layers of skin, fat, and muscle directly to the root of pain causing inflammation.

The present invention includes several interrelated embodiments including a composition in the form of an oil, a composition in the form of a cream, a system that includes a cream and an oil, a method of manufacturing a cream, a method of manufacturing an oil, and a method of using a system that includes an oil and a cream. Basic aspects of the compositions and methods are discussed immediately below, while a discussion containing more information regarding individual components follows.

In an exemplary embodiment, the invention is an oil that includes at least one transducer, a solvent, at least one fibrinolytic agent, and at least one of a pain relief agent and an anti-inflammatory agent. In some embodiments of the oil, both a pain relief agent and an anti-inflammatory agent are present. In some embodiments, the oil also includes at least one of a cooling component, a circulation enhancing component and a joint or muscle relief component. Embodiments can include a transducer, at least one fibrinolytic agent, at least one pain relief agent at least one anti-inflammatory agent, at least one cooling component, at least one circulation enhancing component and at least one joint or muscle relief component. Without being bound by theory, it is believed that the transducer promotes absorption or permeation of the composition into the skin. Some components of the transducer may also function as anti-inflammatory agents or skin conditioning agents. The fibrinolytic agent can enhance penetration of the composition into a muscle or joint, particularly when the muscle or joint has accumulated scar tissue or a build up of fibrin as a result of insult or injury. The remaining active component(s) are clinically proven to relieve pain and/or enhance healing due to known properties as described herein. However, the combination of ingredients with a transducer and fibrinolytic to enhance activity, pain relief and healing has not been previously suggested.

The invention also includes a cream that is a combination of the oil with a cream base. The cream base (and thus the cream) includes cetlyated fatty acids and a surfactant, emulsifier or solubilizing agent. The cream base can also include a cooling agent and/or a pain relief agent. In exemplary embodiments the cream base includes the same cooling and/or pain relief agent(s) as the oil and these agents can be present in the same combination and concentration. It is believed that the presence of the cooling agent and/or the pain relief agent in the cream base provides improved mixing of the cream base with the oil, that is, a stable emulsion of the final composition can be more readily achieved. Furthermore, including the cooling agent and/or pain relief agent in the cream base at the same concentration as in the oil allows for variability in the portions of the oil and cream base used to make the cream, all of which result in a consistent concentration of the cooling agent and/or pain relief agent in the final cream. For example, if it is desired to have a concentration of 10% cooling agent in the final cream, the oil and the cream base each contain 10% cooling agent. This is as an alternative to preparing an oil that has 20% cooling agent in equal proportions with a cream that has no cooling agent. Advantages of having the same concentration of cooling agent in both the cream and the oil will become apparent from the methods of use discussed below.

In other embodiments, the invention is a system or kit that includes both a cream and an oil as described above. An advantage of the system is that it provides a number of methods of use for a wide range of pain relief as required by the severity of the pain. For example, for mild pain, the cream of the system need only be applied. This includes a lower concentration of oil components that are not included in the cream base and therefore provides a “milder” version of the composition of combined ingredients. This can be advantageous because some components of the oil, if not attenuated by the presence of cream components such as, for example, cetylated fatty acids, can cause discomfort by, for example, causing excessive heat release in sensitive areas such as the neck and face. If application of the cream alone does not provide sufficient pain relief, the oil can be applied over the cream. This has two effects. First, it increases the concentration of the components from the oil that are not in the cream. Second, and related to the first, because the oil can include fibrinolytic agents and transducers at higher concentration than the cream alone, application of the oil on top of the cream can enhance transport of the cream components, specifically the cetylated fatty acids, through the skin and into the muscle or joint that is experiencing pain. Additional application of the oil to the same area further enhances penetration of the cetylated fatty acids and other components. Moreover, because certain active components, for example the cooling component and the pain relief component, are present in the same concentration in both the oil and the cream, the overall concentration of such components does not change. Finally, for persistent pain, the oil alone can be applied. While application of the oil alone does not provide the benefits of the cetylated fatty acids in the cream base, it provides a higher concentration of active ingredients not present in the cream base, but only in the oil.

Pain Relief Component

Many patients with localized pain due to arthritis, bursitis, sprain or muscle strain, bruises or hematomas cannot tolerate conventional non-steroidal anti-inflammatory drugs (NSAIDs) particularly when administered orally. In addition, topical administration of conventional NSAIDs has largely been ineffective because only a therapeutically ineffective amount of the drug can penetrate the skin. In addition, indications such as acne, psoriasis and eczema are typically refractory to topical or oral administration of NSAIDs. Accordingly, there is a need for compositions that include pain relief agents other than NSAIDs particularly in a topically applied formulation. There is also a need for an anti-inflammatory composition that is effective in treating a wide variety of inflammatory conditions by topical application of the composition. The anti-inflammatory composition should not have the side effects associated with prior art NSAIDs. The present invention addresses the needs in the prior art by providing a pain relief and anti-inflammatory composition that avoids such drawbacks.

Exemplary pain relief components include capsaicin, capsaicinoids and capsaicin analogues and derivatives. Examples of capsaicinoids include dihydrocapsaicin, nordihydrocapsaicin, homodihydrocapsaicin, homocapsaicin, and nonivamide. Capsaicin analogues include resineferatoxin, hydroxyphenylacetamindes having capsaicin like structures, capsaicin compounds having a modified amide group, e.g. where —NHC(O)— is replaced by —C(O)NH—, —NHC(O)O—, —NHC(S)—, —C(S)NH—, —NHC(O)NH—, —NHC(S)NH—, —NHS(O)₂—, and the like. Attention is hereby drawn to the following publications for their disclosure of capsaicinoid or a capsaicin analogues and derivatives: U.S. Pat. Nos. 4,424,205, 5,962,532, 5,290,816, and 4,812,446, and K. Kobata et al. “Novel Capsaicinoid-like Substances Capsiate and Dihydrocapsiate, from the Fruits of a Nonpungent Cultivar, CH-19 Sweet, of Pepper (Capsicum annum L.)”, J. Agric. Food Chem. 46(5), 1695-1697, (1998). The capsaicin can be white capsaicin, red capsaicin or combinations thereof. Exemplary compositions include up to 1.5% capsaicin, for example, up to 1.5% capsaicin, from about 0.001% to about 0.75% capsaicin. Exemplary embodiments contain about 0.025% capsaicin. Capsaicin and its analogues and derivatives work by depleting Substance P and disabling the transmission of pain messages to the brain for meaningful long-term relief.

Other pain relief agents which may be used alone or in combination with capsaicin, capsaicinoids or capsaicin analogues and derivatives include, but are not limited to, lidocaine, aloe vera, MSM, Willow Bark (salix alba) Extract, menthol, glucosamine, chondroitin, ginger, gotu kela, ginkgo, juniper, arnica extract, feverfew extract, St. John's wort extract, kava kava extract, nettle leaf, acetylsalicylic acid, Bala, Black cohosh, meadow sweet, passion flower, quercetin, salicinum, wild yam, wintergreen, wood betony, wormwoode, and essential oils such as limonene, lavender, Ylang, Coriander, Tea Tree, Valerian Root, evening primrose, olive leaf, chamomile, emu.

In exemplary embodiments, the pain relief agent is an active ingredient present in the oil component of the composition and thus is also present in the cream. In some embodiments, one or more of the pain relief components is present in the cream base. In exemplary embodiments, one or more of the pain relief components is present in the cream base at the same concentration as in the oil so that the concentration in the final cream is the same as the concentration in the oil.

Anti-Inflammatory Component

A broad range of anti-inflammatory agents can be used in compositions of the invention. In general, any anti-inflammatory agent will be present as part of the oil, although they may also be part of the cream base or in the cream base alone. Exemplary anti-inflammatory agents include boron, Boswella, for example Boswella serrata, or an extract thereof, burdock root, or an extract thereof, for example bur oil, calendula or an extract thereof, cat's claw, citric bioflavaoids, curcumin, for example white curcumin, devil's claw, cetyl esterified fatty acids, ginger or an extract thereof, olive oil, for example extra virgin olive oil, or an extract thereof, safflower oil or an extract thereof, vitamin D3,vitamin E willow bark herb extract, wintergreen or wintergreen oil and combinations thereof. The amount of anti-inflammatory agent is variable depending upon its identity and strength. For example, when the anti-inflammatory agent is a naturally occurring oil, such as wintergreen oil, or contains a naturally occurring oil, it can be present in relatively large amounts such as up to 50% of the oil, for example from about 0.001% to about 50% of the oil; more than 10%, about 10% to about 40% up to about 40% of the oil or about 30% of the oil. More potent anti-inflammatory agents can be present in much lower quantities, for example, up to about 10%, or from about 0.001 to about 10%, up to about 5.0%, up to about 4.0%, up to about 3.0%, up to about 2.0%, up to about 1.0%; or in an amount of about 5.0%, about 4.0%, about 3.0%, about 2.0%, or about 1.0%. Persons skilled in the art will recognize the amount of ingredient that is needed for a particular anti-inflammatory agent, and amounts used in the present invention are generally similar to what is known in the art.

Fibrinolytic Component

As a result of injuries or insults that cause pain, particularly in the area of joints, bodies react by sending white blood cells to the area, causing inflammation and swelling. This releases fibrin, a natural substance in that helps heal wounds. However, release of fibrin seals off the area with a protective mesh. Even when able to penetrate the skin, prior art topical pain relief and anti-inflammatory formulations are unable to pass through the fibrin mesh, and therefore are unable to directly treat or provide therapeutic relief at the site of the injury. Although the affected area generally heals, sometimes fibrin continues to build around the site, then hardens, causing further inflammation and pain. The present invention can thus include a proteolytic agent, for example a fibrinolytic, to promote delivery of the actives through the fibrin directly to the actual area of inflammation and associated pain or to aid in the decomposition of fibrin to further reduce pain and inflammation.

An exemplary fibrinolytic for use in the present composition is bromelain, although other fibrinolytic and proteolytic components can be used. Bromelain is a protease composition that is isolated from pineapple. Bromelain has been reported to have anti-inflammatory activity when administered orally or parentally (Taussig, S. J. “The mechanism of the psychological action of Bromelain”, Medical Hypotheses, 6;99-104, 1980). Bromelain has been reported to be an anti-inflammatory agent, an inhibitor of platelet aggregation, an agent that increases proteolytic and fibrinolytic activity in blood, and as a selective prostaglandin inhibitor. Bromelain has been administered by injection and has been reported to be effective after oral administration. However, because bromelain is a macromolecule, it typically cannot be administered trans-dermally using prior art formulations. Compositions according to the present invention that include transducers as described herein are believed, based on efficacy, to effectively promote transdermal delivery of fibrinolytics such as bromelain.

The fibrinolytic component is generally present in the oil. The fibrinolytic can be present in amounts up to about 10%, for example in amounts from about 0.001% to about 10%. In other exemplary embodiments, the fibrinolytic component is present in the oil in an amount of from about 1.0% to about 4.0%, for example about 2.5%, or in an amount of from about 2.0% to about 8.0%, for example about 5.0%. In other embodiments, the fibrinolytic can be present in amounts up to about 5.0%, for example in amounts from about 0.01% to about 5.0%. In other exemplary embodiments, the fibrinolytic component is present in the oil in an amount of from about 1.0% to about 2.0%, for example about 1.5%, or in an amount of from about 2.0% to about 4.0%, for example about 3.0%. The fibrinolytic can be present in this amount as a single fibrinolytic component, a combination of fibrinolytic components, a single proteolytic component, a combination of proteolytic components or a mixture of one or more fibrinolytic components and one or more proteolytic components

Cooling Component

Compositions of the invention can include a cooling component. This component can decrease burning and discomfort associated with capsaicin and pain perception.

Exemplary cooling agents include, but are not limited to at least one agent selected from the following: Menthone, glycerin acetal, menthyl lactate, ethyl menthane carboxamide, methyl diisopropyl propionamide, peppermint oil, menthol and menthoxypropanediol. Menthol, or its related compounds known in the art, is an exemplary component used in the present formulation as a cooling component for soothing pain relief and as a signal of efficacy.

In exemplary embodiments, the cooling agent is an active ingredient present in the oil component of the composition and thus is also present in the cream. In some embodiments, one or more of the cooling components is present in the cream base. In exemplary embodiments, one or more of the cooling components is present in the cream base at the same concentration as in the oil so that the concentration in the final cream is the same as the concentration in the oil. The cooling agent can be present in an amount up to about 15%, for example from about 0.0001% to about 15%. In exemplary embodiments, the cooling agent is present in an amount of from about 8.0% to about 12%, for example about 10%.

Cetyl Esterified Fatty Acids

Cetyl esterified fatty acids (also referred to herein as cetylated fatty acids) have been clinically proven to absorb readily in order to provide immediate and continuous cumulative pain relief. They enhance cell membrane integrity throughout the body and restore fluids that cushion bones and joints to promote flexibility and mobility. Loss of cell membrane integrity includes uncontrolled or imbalanced inflammation which stiffens cell membranes. This leads to one or more forms of arthritis, bursitis and tendonitis included. Cetyl esterified fatty acids are believed to halt the cascade of inflammation and rapidly restores the body on a cumulative basis. Flexibility is restored and pain is eliminated. The combination of cetylated fatty acids with other components of the present invention provides fast and effective treatment modality in the area of pain management and improves joint function and flexibility. Thus, the presence of cetylated fatty acids not only provides pain relief, but can promote healing.

The invention provides novel and advantageous methods of delivery of cetyl esterified fatty acids. The delivery methods are useful in the treatment of several diseases affecting humans and animals. Cetyl esterified fatty acids are also useful in the treatment of inflammatory conditions that affect joints such as arthritis, juvenile chronic arthritis, chronic arthritis. Diehl and May, (1994) J Pharm Sci 83 (3): 296-299. Cetyl esterified fatty acids can also be used in the treatment of migraine, emphysema, asthma, myofascial pain, arteriosclerosis, joint sports injuries and sprains, insulin dependent diabetes, peripheral vascular disease, carpal tunnel syndrome, cardiomyopathy, chronic fatigue immune dysfunction syndrome, Churg-Strauss syndrome, and psoriasis.

Cetylated fatty acids include, for example, cetyl laureate, cetyl lineoleate, cetyl myristate, cetyl myristoleate, cetyl oleate, cetyl palmitate, cetyl palmitoleate. One or more ceylated fatty acids can be present in the oil, for example in a total amount up to about 1.0%, such as from about 0.001% to about 0.5%, or from about 0.1% to about 0.3% or about 0.2%. As known in the art, cetylated fatty acids can be difficult to solubilize in oil compositions, and so this component should be present in the oil in an the amount that can remain soluble. Cetylated fatty acids are also a component of the cream base and can be the primary component of the cream base used to produce a cream composition according to the invention. As described elsewhere herein, the cream base can also include an emulsifier or surface active agent, and can include the cooling component and/or pain relief component of the oil at a concentration similar to that of the oil.

Transducer

The transducer is used to enhance skin permeation or transdermal delivery of active agents and is believed to “carry” other components of the invention into the skin. The transducer component is thus a compound or composition (i.e. mixture of compounds) that enhances skin permeation. Exemplary transducers that can be used in the present invention include emu oil, and aloe vera, for example aloe vera oil. Preferably, the transducer component is prepared in a very pure form. For example, when natural oils are used, they can be purified in order to remove components, such as high molecular weight and fatty acid components, that resist solubilization in the oil.

The transducer component can be a single component or a blend of multiple components. In some embodiments, the solvent can also function as a transducer component. The transducer can each be present in an amount up to about 40.0%, for example from about 0.001% to about 40.0%, from about 2.0% to about 40.0%, from about 2.5% to about 30% or about 3.5%, about 2.0%, about 2.5%, about 30.0% or about 40.0%. Obviously, as is apparent to persons in the art, in cases where the solvent functions as a transducer component, the amount of transducer component is higher. In some embodiments, one or more transducer components can be present in an amount up to about 10.0%, for example from about 0.001% to about 10.0%, from about 2.0% to about 8.0%, from about 3.5% to about 6.0% or about 5.0%. In some embodiments, one or more transducer components can each be present in an amount of from about 0.001% to about 2.0%, from about 0.5% to about 1.5% or about 1.0%. In still other embodiments, a transducer component can be present in an amount up to about 5.0%, for example from about 0.001% to about 5.0%, from about 2.0% to about 5.0%, from about 2.5% to about 3.5%, about 2.0% or about 2.5%.

Circulation Increasing Component

Compositions according to the invention can include a circulation increasing agent. The circulation increasing component is intended to effectuate increased oxygen uptake by increasing blood supply at the point of pain, and can also provide better penetration of the actives to the skin and nerves. Many anti-inflammatory agents are also effective at increasing circulation, such as, for example, wintergreen oil. Other circulation increasing agents, which may be used include but are not limited to lime tree extract, marigold extract, feverfew extract, St. John's wort extract, arnica extract, chamomile oil, nettle extract, marigold extract, grape seed extract, witch hazel extract, arjuna, Bala, benzoin, bilberry, black pepper, blue gum eucalyptus, MSM, blue bottle extract, coriander oil, blue vervain, borneol, butcher's broom, cayenne, cypress, geranium, ginger, gingko, ginseng, grape seed, proanthocyanidin extract, Hawthorn, L-arginine, lemon, lemon grass, limonene, linden flowers, niaoili, oat straw, orange blossom, passion flower, thyme, violet, Per balsam, pine, prickly ash bark, rose oils, rosemary, Spanish sage, spruce, tea tree oil, white birch, yohimbe and combinations thereof.

Joint Or Muscle Soothing Components and Joint or Membrane Lubricants

The present invention can further include a joint or muscle soothing component that can also have anti-inflammatory effects. Cetylated fatty acids can act as joint lubricants and thus provide joint or muscle soothing properties. An exemplary joint or muscle soothing anti-inflammatory complex includes glucosamine (for example as either sulfate or hydrochloride complex, and either sodium or potassium salt), ginger root extract, MSM, Polygonum cuspidatum (Mexican Bamboo) extract, Aloe barbadensis leaf, and Salix alba (white willow) bark extract. Additional commercial products that provide joint or muscle soothing properties or act as joint or membrane lubricants include, for example, UC-II® (InterHealth Nutraceuticals, Inc. of Benicia, Calif.), natural eggshell membrane (such as NEM®, a composition that includes the glycosaminoglycans—chondroitin and hyaluronic acid, plus collagen and other proteins available from ESM Technologies, Carthage Miss.), as well as similar products that may be available from additional sources.

Cream/Cream Base

The cream described herein is prepared by mixing the oil with a cream base.

The cream can be prepared by mixing the oil and cream base in a range of proportions, for example from about 10:1 oil to cream base to about 1:10 oil to cream base or from about 1:3 oil to cream base to about 3:1 oil to cream base. Exemplary embodiments of the cream are prepared from about 90% cream base and about 10% oil, about 75% cream base and about 25% oil, about 64% cream base and about 33% oil, about 60% cream base and about 40% oil, about 50% cream base and about 50% oil, about 40% cream base and about 60% oil, about 33% cream base and about 64% oil, about 25% cream base and about 75% oil, or about 10% cream base and 90% oil. Other proportions can also be used.

As previously disclosed, a primary component of the cream base can be a cetyl esterified fatty acid or a mixture of cetyl esterified fatty acids. The mixture can include, for example, cetyl laureate, cetyl lineoleate, cetyl myristate, cetyl myristoleate, cetyl oleate, cetyl palmitate, and cetyl palmitoleate. The amount of each individual cetyl esterified fatty acid can be varied to achieve a desired texture or effect. In an exemplary embodiment, the prevalence of each cetyl esterified fatty acid is adjusted so that the mixture contains, from most prevalent to least prevalent:
cetyl palmitate>cetyl laureate>cetyl myristate>cetyl lineoleate≈cetyl myristoleate>cetyl oleate>cetyl palmitoleate

In an exemplary embodiment, the ration of cetylated fatty acids is about 4 parts cetyl palmitate, 2.5 parts cetyl laureate, 2.25 parts cetyl myristate, 2 parts cetyl lineoleate, 2 parts cetyl myristoleate, 1 parts cetyl oleate and 1 part cetyl palmitoleate.

As described previously, the cream base can also include one or more components that is also present in the oil, such as the cooling component and pain relief component. The component can be present in the same concentrations as in the oil so that the final concentration in the cream is the same as in the oil, assuring that whether the oil or cream is applied alone or the oil and cream applied sequentially, the concentration of these components actually applied will be the same. In order to prepare a cream base that can be mixed with the oil components and, eventually with the oil, it may be necessary to add an emulsifying agent or surfactant. The particular emulsifying agent or surfactant and the amount used should be selected such that, after mixing, the cream base is a stable emulsion. Furthermore, the particular emulsifying agent or surfactant and the amount used should be selected such that, after mixing with the oil, a stable emulsion (cream) is formed. Exemplary emulsifying agents or surfactants include cetostearyl alcohol, glycerine, lecithin organanol, or other liposome forming component, polyethylene glycol, polysorbate 80, propylene glycol, and triethanolamine (TEA), although other emulsifying agents, surfactants and the like that stabilize emulsions may be used.

Other Components

Compositions according to the invention can include additional components to provide additional physically beneficial effects, as well as for other purposes. Exemplary embodiments of the composition include muscle membrane stabilizers such as magnesium compounds, such as, for example, magnesium chloride, dibasic magnesium, magnesium oxide and ionized magnesium. The oil generally utilizes a solvent to solubilize the various components. Any solubilizing solvent that provides a stable oil is acceptable. The solvent can itself have beneficial properties as will be known to persons skilled in the art. In exemplary embodiments, the solvent is dimethyl sulfoxide, for example USP grade dimethyl sulfoxide. Other components can also include preservatives or pH modifiers. Other solvents may be added as necessary during processing, but are generally removed during the process

Other non-active (i.e. non-active with respect to pain relief activity) ingredients can include antimicrobial components such as, for example, lavender oil, bisabolol, limonene and lemon oil; skin protectants such as, for example, aloe vera, mineral oil, lanolin, and shea butter; thickeners or texture modifiers such as, for example, cetostearyl alcohol, parabens, for example propyl paraben, methyl parabens, etc.; preservatives and antioxidants such as, for example, disodium EDTA, parabens, for example propyl paraben, methyl parabens, etc., milk thistle, and bioflavinoids, for example citric bioflavinoids; fragrances such as, for example, tea tree oil; and pH adjustors such as, for example, citric acid. Other ingredients known in the art to serve such functions may also be included.

The present invention is a composition for treatment for the inhibition of pain.

In exemplary embodiments, the formulation is a combination all natural clinically proven natural extracts with anti-inflammatory, substance P blockers, muscle membrane stabilizers, and proprietary blend of cetyl esterified fatty acids. These could comprise of cetyl laureate, cetyl lineoleate, cetyl myristate, cetyl myristoleate, cetyl oleate, cetyl palmitate, cetyl palmitoleate. The invention relates to novel and advantageous methods of delivery of cetyl esterified fatty acids. The delivery methods are useful in the treatment of several diseases affecting humans and animals. Cetyl esterified fatty acids are also useful in the treatment of inflammatory conditions that affect joints such as arthritis, juvenile chronic arthritis, chronic arthritis. Diehl and May, (1994) J Pharm Sci 83 (3): 296-299. Cetyl esterified fatty acids can also be used in the treatment of migraine, emphysema, asthma, myofascial pain, arteriosclerosis, joint sports injuries and sprains, insulin dependent diabetes, peripheral vascular disease, carpal tunnel syndrome, cardiomyopathy, chronic fatigue immune dysfunction syndrome, Churg-Strauss syndrome, and psoriasis.

Exemplary active and inactive ingredients useful in the invention thus include, for example, willow bark herb extract, wintergreen, aloe vera, tocopherol acetate mix, carbopol 940NF, limonene, vitamin D3, vitamin E, panax ginseng, bisabolol, SalCool™, ginger, cinnamon leaf, dandelion, devil's claw, cat's claw, emu oil, mineral oil, folic acid, capsaicin, disodium EDTA, magnesium, magnesium dibasic, magnesium chloride, ionized magnesium, triethanolamine (TEA), polysorbate 80, cetostearyl alcohol, polyethylene glycol, propylene glycol, propylene paraben, methyl paraben, glucosamine sulfate, USP dimethyl sulfoxide, MSM, bromelain, extra virgin olive oil, calendula boswella serrata, menthol, cetyl esterified fatty acids (cetyl laureate, cetyl lineoleate, cetyl myristate, cetyl myristoleate, cetyl oleate, cetyl palmitate, cetyl palmitoleate), limonene, glycerine, lecithin organanol, liposome, milk thistle, arnica, lavender, lemon, citric acid, burdock root, boron, lanolin, isopropyl myristate, myritic acid, fatty acids 3-6-9 unsaturated fatty acids, nem oil, marigold, citric bioflavaoids, red clover, cetearyl alcohol, lanolin alcohol, sterayl alcohol, cetyl alcohol, shea butter, safflower oil, willow bark, ginger, peppermint or peppermint oil, emu oil and tea tree oil.

Shown below are exemplary ingredients that can be used in formulations according to the invention, such as creams, oils, gels, salves, sprays, powder, balms, liniment, and ointments, including approximate maximum amounts and exemplary ranges. It is not necessary that all of the above ingredients be added. Thus, the amounts recited are exemplary and represent the quantities represented are exemplary only for those compositions that include the particular component. Accordingly, the amounts recited are non-limiting and a person skilled in the art can use these ingredients and others for a satisfactory purpose. In addition to the above ingredients, compounds typically used in the art, for example preservatives and pH modifiers, may be incorporated.

| | Approximate | | |
|---|---|---|---|
| | Maximum | | |
| Component | Amount (%) | Exemplary ranges (%) | |
| Willow bark herb extract | 20 | 0.0001-0.40 | 0.001-0.35 |
| Wintergreen, for example | 60 | 3.00-30 | 9.00-26.40 |
| as an oil | | | |
| Aloe vera, for example as | 45 | 2.00-30 | 6.00-30 |
| an extract or oil | | | |
| Tocopherol acetate mix | 2 | 0.15-1.50 | 0.45-1.50 |
| Carbopol 940NF | 25 | 1.00-20 | 3.00-15 |
| Vitamin D3 | 9 | 0.050-1.80 | 0.150-1.80 |
| Vitamin E | 9 | 0.050-2.0 | 0.150-1.75 |
| Panax ginseng, for | 15 | 0.150-2.0 | 0.450-1.75 |
| example as an extract or | | | |
| oil | | | |
| Bisabolol | 3 | 0.050-1.0 | 0.150-0.90 |
| Salcool ™ | 30 | 0.050-1.0 | 0.150-0.90 |
| Ginger, for example as an | 15 | 0.002-1.25 | 0.006-1.20 |
| extract or oil | | | |
| Cinnamon, for example | 10 | 0.002-1.25 | 0.006-1.20 |
| as an extract or oil such | | | |
| as for example cinnamon | | | |
| bark extract | | | |
| Dandelion, for example | 10 | 0.0001-0.40 | 0.001-0.36 |
| as an extract or oil | | | |
| Devil's claw | 20 | 0.0001-0.40 | 0.001-0.36 |
| Cat's claw | 20 | 0.0001-0.40 | 0.001-0.36 |
| Emu oil | 50 | 0.20-50 | 0.60-48 |
| Mineral oil | 10 | 0.10-2.50 | 0.30-2.40 |
| Folic acid | 5 | 0.002-0.15 | 0.006-0.12 |
| Polysorbate 80 | 23 | 0.02-20 | 0.06-20 |
| Cetostearyl alcohol | 5 | 0.04-4.0 | 0.12-3.60 |
| Capsaicin (Red, white or | 1 | 0.002-0.35 | 0.006-0.30 |
| mixture) | | | |
| Disodium EDTA | 10 | 0.002-0.15 | 0.006-0.12 |
| Magnesium | 5 | 0.002-1.0 | 0.006-0.95 |
| Magnesium dibasic | 5 | 0.002-1.0 | 0.006-0.95 |
| Magnesium chloride | 5 | 0.002-1.0 | 0.006-0.95 |
| Ionized magnesium | 5 | 0.002-1.0 | 0.006-0.95 |
| Propylene glycol | 10 | 0.20-7.5 | 0.60-6.0 |
| Triethanolamine (TEA) | 4.5 | 0.20-7.5 | 0.60-6.0 |
| Polyethylene glycol | 10 | 0.20-7.5 | 0.60-6.0 |
| Glucosamine sulfate | 10 | 0.002-1.15 | 0.006-1.08 |
| USP dimethyl sulfoxide | 65 | 5.0-50 | 15.0-48 |
| MSM | 25 | 0.002-4.5 | 0.006-3.0 |
| Bromelain | 20 | 0.002-4.5 | 0.006-3.0 |
| Peppermint Oil | 10 | 0.001-2.50 | 0.001-2.10 |
| Extra virgin olive oil, for | 22 | 0.50-7.50 | 1.75-7.20 |
| example as an extract or | | | |
| oil | | | |
| Calendula, for example | 30 | 0.34-6.0 | 1.02-5.40 |
| as an extract or oil | | | |
| Boswella serrata, for | 10 | 0.10-2.50 | 0.30-2.10 |
| example as an extract or | | | |
| oil | | | |
| Menthol | 35 | 7.5-25 | 8.0-20 |
| Cetylated fatty acids | 90 | 0.15-90 | 25-90 |
| Glycerine | 7.5 | 0.10-2.0 | 0.30-1.8 |
| Lecithin organanol | 15 | 0.10-2.0 | 0.30-1.80 |
| Milk thistle, for example | 7.5 | 0.17-2.1 | 0.51-2.0 |
| as an extract or oil | | | |
| Arnica, for example as an | 25 | 0.10-10 | 0.30-8.40 |
| extract or oil | | | |
| Lavender, for example as | 25 | 0.10-10 | 0.30-8.4 |
| an extract or oil | | | |
| Lemon, for example as an | 20 | 0.10-15 | 0.30-13.2 |
| extract or oil | | | |
| Burdock root, for | 7.5 | 0.007-1.0 | 0.021-0.96 |
| example as an extract or | | | |
| oil | | | |
| Boron | 3 | 0.007-1.0 | 0.021-0.96 |
| Lanolin | 7.5 | 0.10-2.5 | 0.30-2.40 |
| Isopropyl myristate | 5 | 0.10-4.0 | 0.30-3.6 |
| Myritic acid | 5 | 0.10-2.0 | 0.30-1.8 |
| Fatty acids, for example | 50 | 0.40-15 | 1.20-12 |
| 3,6,9-unsaturated fatty | | | |
| acids | | | |
| Nem oil (NEM ®) | 3 | 0.04-1.5 | 0.12-1.2 |
| Marigold, for example as | 12 | 0.0001-1.0 | 0.001-0.84 |
| an extract or oil | | | |
| Citric bioflavaoids | 15 | 0.002-4.0 | 0.006-3.6 |
| Red clover, for example | 5 | 0.002-4.0 | 0.006-3.6 |
| as an extract or oil | | | |
| Cetyl alcohol | 2.5 | 0.10-2.5 | 0.30-2.40 |
| Lanolin alcohol | 20 | 0.60-8.0 | 1.80-6.0 |
| Stearyl alcohol | 20 | 0.6-8.00 | 1.80-6.0 |
| Cetyl alcohol | 10 | 0.0001-2.5 | 0.0001-2.4 |
| Shea butter, for example | 20 | 0.35-3.00 | 1.05-3.0 |
| as an extract or oil | | | |
| Safflower oil or extract | 20 | 0.35-3.0 | 1.05-3.0 |

In some formulations, the composition can be prepared in the form of a gel.

Gels can be prepared by addition of a gelling agent. Gelling agents can include, for example, polysaccharides or proteins, natural gums, starches, pectins, agar-agar and gelatin, as well as syntheitc gelling agents. Examples of gelling agents include ammonium alginate, calcium alginate, agar, carrageenan, locust bean gum, pectin and gelatin. In some embodiments, water or a lower alcohol may be needed to form a suitable gel. The composition can also be supplied or applied in the form of salves, sprays, powder, balms, liniment, ointments, wipes, adhesive strips, and transdermal patches.

As described previously, the cream only may be applied or the oil only may be applied. Alternatively, the cream can be applied initially with one or more applications of the oil over the cream. In exemplary embodiments, the method of use includes topical application of the cream to the area experiencing pain. If pain persists, the oil is applied on top of the cream, and the application of oil repeated if pain continues. If the combination of cream and multiple applications of oil is insufficient to relieve pain, the oil can be used alone. The oil and/or cream can be applied to painful inflamed joints directly. The composition can be applied to one side of joint, around the joint, or, if severe pain exists, approximately equal amount of the composition can be applied to each side of the joint. The amount of composition that is applied to skin is not critical to invention. It is important that composition is thoroughly rubbed into skin and each person will determine how much of the anti-inflammatory composition they will need.

In some embodiments, the composition (in any suitable form) may be applied to an applicator that is itself applied to the skin. The applicator may be, for example, a wipe or pad. The pad can alternatively be attached to an adhesive strip that can be attached to the skin. In this embodiment, the adhesive strip can be arranged to form a patch that is applied to the skin for an extended period of time.

Administering compositions according to the present invention show beneficial effects not achieved with other formulations. For example, the present invention provides easy and safe methods of administering an effective anti-inflammatory and pain reliever composition topically directly at site of inflammation. Present invention is useful for various forms of inflammation indications including arthritis, neuralgia, rheumatoid arthritis, sprains, bursitis, myositis, carpel tunnel syndrome. In an exemplary embodiment, 2 cc of the composition was applied for back pain clinical trials for 2-3 days. In another exemplary embodiment, 2 cc of the composition was applied for knee pain clinical trials for about two or four weeks. The compositions were able to provide pain relieving effects for from 6 hours to 12 hours.

Compositions in the form of an oil can be prepared by solubilizing all ingredients, for example in a blend of purified emu oil or pure aloe vera. Specific steps will be described in the following section.

### EXAMPLES

### Example 1

### Preparation of an Exemplary Embodiment

A cream formulation was prepared as follows.
1) Prephased capsaicin in emu oil and aloe vera, and wintergreen were mixed for 15 minutes. About half of the capsaicin in 1% methanol was added until solubilized then the remaining capsaicin added at a rate to minimize phase separation.
2) Prephased menthol previously dissolved in aloe vera and emu oil was added to the solution and heated to 45-50° C. until methanol evaporated and then stirred for 20 minutes.
3) Prephased dimethyl sulfoxide with wintergreen oil and mixed for 15 minutes.
4) Homogenized main solution for 20 minutes.
5) Added menthol, heated to between 40 and 45° C., to main solution.
6) Prephased bromelain in emu oil and aloe vera in prephased Step 3 solution, then added to main solution.
7) Homogenized main solution for 20 minutes.
8) Prephased boswella serrata extract in emu oil, added to solution and mixed with heat of between 20-30° C. for 10 minutes, set aside.
9) Added first portion of lecithin to main base solution.
10) Prephased wintergreen in portion of limonene continuing in increments in aloe vera and emu oil and continued to stir for 5 minutes and set aside.
11) Prephased ginseng in portion of Step 8 solution and stirred before adding to solution.
12) Homogenized main solution for 20 minutes.
13) Prephased calendula in aloe vera and emu oil heated to 45-55° C., and stirred then added to main solution.
14) Combined arnica and magnesium with Step 10, added to solution first and mixed for 10 minutes before adding the rest to solution, mixed for another 20 minutes.
15) Homogenized main solution for 20 minutes.
16) Prephased vitamins D3 and E in portion of solution then added willow bark, tea tree oil and cinnamon bark oil to this solution and heated together at 40-45° C. then added this combined mixture to the main solution.
17) Let main solution cool down to about 30° C.
18) Added a portion of the polysorbate 80 to main base solution.
19) Homogenized main solution for 20 minutes.
20) Prephased milk thistle, MSM, citric bioflavanoids, and limonene in aloe vera and emu oil then added to main solution.
21) Prephased lavender, lemon, ginger together in aloe vera and emu oil and let it sit for 20 minutes before adding to main solution.
22) Prephased cat's claw, devil's claw, and burdock root each separately in aloe vera and emu oil and stirred until solubilized before adding to main solution.
23) Homogenized main solution for 20 minutes.
24) Prephased magnesium and added to main solution.
25) Prephased with olive oil in aloe vera and emu oil and added to main solution.
26) Homogenized main solution for 20 minutes.
27) Added portion of polysorbate 80 to main solution and mix for 30 minutes.
28) Mixed cetyl myristoleate and purified deionized water with emulsifier blend poly acrylamide C13-14 isoparaffin laurath-7 vegetable glycerin, cetyl oleate, cetyl myristate, cetyl lineoleate, cetyl palmitate, cetyl palmitoleate, cetyl laurate, benzyl alcohol and glyceryl stearate to emulsify, added oil of peppermint, lecithin, PEG100 stearate, dialpha tocopheryl acetate.
29) This mixture was heated to 35-45° C. and kept this way for 30 minutes, then light agitation was introduced and continued for approximately 1 hour.
30) Added cetearyl alcohol, lanolin alcohol, stearyl alcohol, cetyl alcohol and left to stand until it reached room temperature.
31) Placed the cetyl mixture under refrigeration for at least 24 hours at 0-5° C., then mixed well and let stand until reached room temperature and added cetyl ester waxes.
32) At room temperature, added about half of cetylated fatty acids with mineral oil, directly to main base.
33) Added bisabolol and mixed with tocopheral acetate and main base.
34) Homogenized main solution for 20 minutes.
35) Added portion of main solution with cetyl esterified fatty acids, added additional portion of polysorbate 80, fatty acids with co-stat mixing, added lemon to main base and stirred for 30 minutes at 35-40° C.
36) Added the remainder of polysorbate 80 to main base and mixed for 15 minutes.
37) Added the rest of emu oil and aloe vera and emu oil to main base and mixed for 30 minutes.
38) Added disodium EDTA and remainder of cetylated fatty acid solution to main base and heated to 35-40° C.
39) Homogenized the entire main base solution for 2 hours.
40) Prephased citric acid to main base solution and mixed for 2 hours and brought solution to room temperature.

Oil formulations can be prepared in a similar way, but include lower amounts of cetylated fatty acids and emulsifier, surfactant and/or solublizing agent. A cream base can also be prepared similarly by omitting ingredients desired only in the oil.

### Example 2

### Effectiveness in the Treatment of Golfers with Back Pain

Background: Low back pain is a very common condition among golfers and has functional implications on the golf course. Due to the poor tolerance and systemic side effects of oral analgesics, there has been a recent interest in topical preparations for the treatment of back pain.

Objective: The purpose of this study is to determine the effectiveness of a topical oil according to the invention in the treatment of back pain in golfers.

Methods: This was a prospective double-blind randomized study. Twenty-eight golfers were evaluated (15 males, 13 females) with average age of 62.5 years (range 44-80 years). Inclusion criteria were a minimum of a three month history of non-specific back pain which increased with golf. Golfers with prior back surgery or leg pain were excluded.

Group I (14 golfers) had 2 cc of the topical oil according to the invention applied day one to the low back fifteen minutes before a round of golf. On day two, they had 2 cc of placebo applied to the low back. Group II (14 golfers) had 2 cc of placebo applied day one to the low back and had 2 cc of the topical oil according to the invention applied on day two.

Efficacy was measured utilizing a numeric pain rating score (0-10) and a patient satisfaction survey (rated as either poor, fair, good or excellent). Wilcox signed rank was used for statistical analysis.

Results: The treatment arm had a pre-golf pain score of 1.35 and a post-golf score of 2.78. The placebo arm had a pre-golf pain score of 1.32 and a post-golf score of 4.79. Comparing the treatment arm post-golf scores to placebo, there was a statistically significant difference (p=0.002). Comparing each individual's post-golf pain score for the treatment arm versus placebo, 18 out of 28 had statistically significant pain reduction (64%). Patient satisfaction of good or excellent was reported by 20 out of the 28 golfers (71%). The tolerance of the oil was excellent with no skin reaction or adverse effects.

Conclusions: The topical oil according to the invention was superior to placebo in the treatment of low back pain in golfers. There were no adverse effects and a majority of the golfers reported good or excellent satisfaction with its use during sporting competition.

### Example 3

### Effectiveness in the treatment of Tennis Players with Knee Pain

Objective: To determine the efficacy of the topical composition of the invention compared to placebo for tennis players who have increased knee pain with tennis

Design: Prospective, double-blinded, randomized study.

Participants: Tennis players with history of increased knee pain with tennis

Twenty-two tennis players were evaluated (13 females, 9 males) with average age of 61.2 years (range 37-77 years).

Interventions: Group I (11 players) had 2 cc of the topical composition according to the invention applied for first two weeks to the knee fifteen minutes before tennis The next two weeks, Group I had 2 cc of placebo applied to the knee. Group II (11 players) had 2 cc of placebo applied to the knee for the first two weeks and had 2 cc of the topical composition according to the invention applied to the knee for the next two weeks.

Main Outcome Measures: Numeric pain rating score (0-10) and patient satisfaction rated as either poor, fair, good or excellent were measured as well as medication use. Wilcox signed rank was used for statistical analysis.

Results: The treatment arm had pre-application pain score of 5.2 on the average (SD 1.2) and a post-application score of 1.6 (SD 1.1) which was statistically significant (p=0.003). Comparing each individual's post-treatment pain score for the treatment arm versus placebo, 16 out of 22 (73%) reported satisfaction of good or better. Anti-inflammatory use was reduced by 37 percent (p=0.005) over the one month time period in the treatment group. The placebo group had pre-application pain score of 5.1 on the average (SD 1.3) while having post-application of placebo score of 4.1 (SD 1.1) which was not statistically significant (p=0.09). Patient satisfaction of good or better was reported by 8 out of 22 (36%). Anti-inflammatory use was reduced by 7 percent which was not statistically significant (p=0.12).

Conclusion: The topical composition according to the invention is superior to placebo at reducing knee pain and anti-inflammatory usage in tennis players.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A pain relief composition comprising a transducer, a fibrinolytic, at least one of a pain relief agent or an anti-inflammatory agent.

2. The composition of claim 1, wherein the pain relief agent comprises capsaicin.

3. The composition of claim 1, wherein the fibrinolytic is bromelain.

4. The composition of claim 1, further comprising at least one of a cooling component, a circulation increasing component, a joint or muscle soothing component and a muscle membrane stabilizer.

5. The composition of claim 1 in the form of an oil, cream, gel, salve, spray, powder, balm, liniment, or ointment.

6. The composition according to claim 1, wherein the composition is a cream and further comprises at least one cetyl esterified fatty acid and an emulsifier or surfactant.

7. The composition of claim 6, further comprising a cooling agent.

8. The composition according to claim 1, wherein the composition is an oil and further comprises a solvent.

9. The composition of claim 1, wherein the composition comprises a cooling agent and the cooling agent is menthol.

10. A method of making a pain relief composition comprising preparing an oil, separately preparing a cream base and combining the oil with the cream base.

11. The method of claim 10 wherein the oil comprises a transducer, a fibrinolytic, at least one of a pain relief agent or an anti-inflammatory agent and a solvent and the cream base comprises at least one cetyl esterified fatty acid and an emulsifier or surfactant.

12. The method of claim 10, wherein the cream base further comprises a cooling agent.

13. The method of claim 10, wherein the oil and the cream base each comprise a pain relief agent and the concentration of pain relief agent in the cream base and the concentration of pain relief agent in the oil are about the same.

14. The method of claim 10, wherein the oil and the cream base each comprise a cooling agent and the concentration of cooling agent in the cream base and the concentration of pain relief agent in the oil are about the same.

15. A pain relief system comprising an oil and a cream, wherein the oil comprises a transducer, a fibrinolytic, at least one of a pain relief agent or an anti-inflammatory agent and a solvent and the cream comprises at least one cetyl esterified fatty acid and an emulsifier or surfactant.

16. The pain relief system of claim 15, comprising a kit, said kit comprising a first container having contained therein the oil and a second container having contained therein the cream.

17. A method for relieving pain comprising topical application of a composition that comprises a transducer, a fibrinolytic, at least one of a pain relief agent or an anti-inflammatory agent, wherein said composition is in the form of an oil or a cream.

18. The method of claim 17, said method comprising topical application of an oil and topical application of a cream.

19. The method of claim 18, wherein the cream is applied to an area first and the oil is applied to the same area after the cream is applied.

20. The method of claim 17, wherein the composition further comprises comprising at least one of a cooling component, a circulation increasing component, a joint or muscle soothing component and a muscle membrane stabilizer.
